(19) United States
(12) Patent Application Publication
(10) Pub. No.: US 2022/0066556 A1
(43) Pub. Date: Mar. 3, 2022

(54) FORCE FEEDBACK DISPLAY METHOD AND DEVICE FOR AN INTERVENTIONAL SURGICAL ROBOT

(71) Applicant: BEIJING WEMED MEDICAL EQUIPMENT CO., LTD., Beijing (CN)

(72) Inventor: Tao Huang, BEIJING (CN)

(21) Appl. No.: 17/213,458

(22) Filed: Mar. 26, 2021

Related U.S. Application Data
(63) Continuation of application No. PCT/CN2021/073706, filed on Jan. 26, 2021.

(30) Foreign Application Priority Data
Sep. 1, 2020 (CN) 202010904421.2

(51) Int. Cl. G06F 3/01; A61B 34/30; G06F 3/0484

(52) U.S. Cl. CPC G06F 3/016; A61B 2090/064; G06F 3/0484; A61B 34/30

(57) ABSTRACT

A force feedback display method and device for an interventional surgical robot are disclosed. In the same display screen, five curve display areas of real-time force measuring curve, a text display area and a numerical display area are configured. According to the force data fed back by the guide wire during propulsion or withdrawal, the real-time force measurement curve can be displayed in the display area of the five curves in real time. The numerical display area displays the force data fed back by the current guide wire in advance or withdrawal in real time. The outer circumference of the five curve display areas is provided with a frame. By observing the high and low positions of the curve, we can clearly see the normal, warning and dangerous states. It can also predict the imminent risk through the trend of curve change, so as to ensure the safe operation.

### CROSS-REFERENCE TO RELATED APPLICATIONS

This application is a continuation of International Patent Application No. PCT/CN2021/073706 with a filing date of Jan. 26, 2021, designating the United States, now pending, and further claims priority to Chinese Patent Application No. 202010904421.2 with a filing date of Sep. 1, 2020. The content of the aforementioned applications, including any intervening amendments thereto, are incorporated herein by reference.

### TECHNICAL FIELD

The disclosure relates to the field of minimally invasive vascular interventional surgery, and more specifically, to a force feedback display method and device of an interventional surgical robot.

### BACKGROUND

Over 30 million people die from cardiovascular and cerebrovascular diseases worldwide every year, accounting for about 30% of all diseases. Among all the population suffering from cardiovascular and cerebrovascular diseases, China accounts for nearly 300 million of all the patients worldwide. Cardiovascular and cerebrovascular diseases have become one of the three major causes of death in human diseases and seriously affect the health and people's daily life.

Minimally invasive cardio-cerebrovascular interventional therapy is the essential treatment for cardio-cerebrovascular diseases. Smaller incision and shorter time to recover from the operation make it surpass the traditional surgery. Cardio-cerebrovascular interventional surgery is a procedure in which doctors manually deliver catheters, guide wires and stents into the patient's body.

However, interventional surgery has two weaknesses. Firstly, X-rays continuously emitted by Digital subtraction angiography (DSA) during the operation, the doctors' physical, concentration and stability drop rapidly, decreasing the accuracy of the operation, and as a result, the escalation of accidents such as vascular intima injury and vascular perforation and rupture caused by improper pushing force occurred, which in the end leads to patient life-threatening. Secondly, the accumulated damage of long-term ionizing radiation raises the risk of doctors suffering from leukemia, cancer, and acute cataracts. It has become an inescapable problem that doctors are infested with radiation which damages their professional life, even limits the development of interventional surgery.

Robot technology will improve the situations. Robot technology is making the surgery more precise and keeps high stability during the operation. It also reduces the radiation damage to the interventional surgeons and the probability of intraoperative accidents. And now the manner of the data transformed from the information acquired from sensors to guide doctors is valid or otherwise in performing surgical operations is a problem that needs to be solved urgently.

Currently, the domestic force feedback for interventional surgical robots has the following problems: (1) The force feedback is only a displayed value, which is of little significance to the doctor's operation guidance. (2) The force change trend cannot be seen, and thus dangerous prediction cannot be made. (3) When the force is large, there is no danger warning.

### SUMMARY

In view of the above problems, the present disclosure proposes a force feedback display method and device for an interventional surgery robot, which is used to solve the problem that there is no suitable force feedback display method for an interventional surgery robot at this stage, and the force feedback data of the robot is inconvenient to use and cannot guide safe operations.

In a first aspect, an embodiment of the present disclosure provides a force feedback display method for an interventional surgery robot. The method includes configuring five curve display areas with real-time force measurement curves in the same display screen, and a text display area and a numerical display area beside the five curve display areas. The five curve display areas includes, from top to bottom, a first danger interval, a first warning interval, a normal interval, a second warning interval, and a second danger interval. The text display areas respectively correspondingly display the five Referential name of the curve display area. The real-time force measurement curve is refreshed and displayed in the five curve display areas in real time according to the force data fed back when the guide wire is advanced or withdrawn. The numerical display area displays real-time feedback force data when the guide wire is advanced or withdrawn. A frame is provided on the outer periphery of the five curve display areas.

Preferably, a warning line is provided between the first danger interval and the first warning interval. Another warning line is provided between the second danger interval and the second warning interval.

Preferably, the refresh frequency of the displayed real-time force measurement curve is configured in advance. The ordinate of the real-time force measurement curve is the force value, and the abscissa is the time.

Preferably, when the guide wire is advanced, the force measurement value displayed in the numerical display area is positive, and the real-time force measurement curve fluctuates between the normal interval, the first warning interval, and the first danger interval. When the guide wire is withdrawn, the force measurement value displayed in the numerical display area is negative, and the real-time force measurement curve fluctuates between the normal interval, the second warning interval, and the second danger interval.

Preferably, the normal interval represents the maximum value range of the force used to ensure the safety of the human body when the guide wire is advanced or withdrawn. The maximum value of the force used to guarantee the safety of human body when the guide wire is advanced or withdrawn is the upper and lower limits of the normal range respectively.

Preferably, when the real-time force measurement curve enters the first warning interval or the second warning interval, the frame is displayed in the first preset mode. When the real-time force measurement curve enters the first danger interval or the second danger interval, the frame is displayed in a second preset manner.

Preferably, the first preset mode and the second preset mode have different present form. The present form includes: different colors, shapes and/or brightness.

In a second aspect, an embodiment of the present disclosure provides a force feedback display device for an interventional surgery robot, which uses the method described in any of the foregoing embodiments for displaying.

The beneficial effects of the above technical solutions provided by the embodiments of the present disclosure include:

An embodiment of the present disclosure provides a force feedback display method for an interventional surgical robot, which presents a smooth real-time force measurement curve in the master-end disclosure of the interventional surgical robot.

By observing the positions of the curve, the doctor can see the current force of the guide wire hold by the robot clearly, and the normal, warning and dangerous states could be observed easily. At the same time, the trend of the curve can also predict the danger that may occur, so as to ensure the safe and effective of the operation. When the force measurement curve exceeds the normal range, the system will change the frame form or display state to remind the doctor to operate carefully.

Other features and advantages of the disclosure will be described in the following description, and partly become obvious from the description, or understood by implementing the present disclosure. The purpose and other advantages of the present disclosure can be realized and obtained by the structures specifically pointed out in the written description, claims, and drawings.

The technical solutions of the disclosure will be further described in detail below through the accompanying drawings and embodiments.

### BRIEF DESCRIPTION OF THE DRAWINGS

The accompanying drawings are used to provide a further understanding of the present disclosure, and constitute a part of the specification. Together with the embodiments of the present disclosure, they are used to explain the present disclosure, and do not constitute a limitation to the present disclosure. In the drawings:

FIG. 1 is a schematic diagram of a hidden force measurement curve of an interventional surgical robot provided by an embodiment of the present disclosure;

FIG. 2 is a schematic diagram of force feedback display of an interventional surgery robot provided by an embodiment of the present disclosure.

In the drawings:

101, force measurement curve; 102, normal interval; 103, first warning interval; 104, second warning interval; 105, first danger interval; 106, second danger interval; 107, first warning line; 108, second warning line; 109, the explanatory text; 110, the measured force value; 111, the frame.

### DETAILED DESCRIPTION OF EMBODIMENTS

Hereinafter, exemplary embodiments of the present disclosure will be described in detail with reference to the accompanying drawings. Although the drawings show exemplary embodiments of the present disclosure, it should be understood that the present disclosure can be implemented in various forms and should not be limited by the embodiments set forth herein. On the contrary, these embodiments are provided to enable a more thorough understanding of the present disclosure and to fully convey the scope of the present disclosure to those skilled in the art.

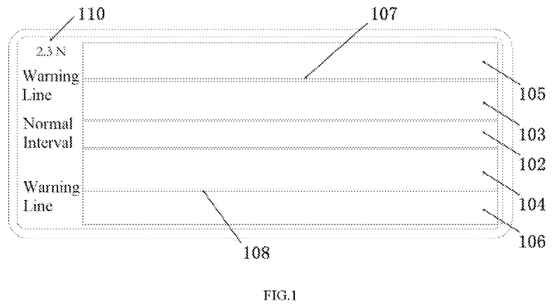

Referring to FIG. 1, an interventional surgical robot force feedback display method provided by an embodiment of the present disclosure includes: five curve display areas of real-time force measurement curves are configured on the same display screen, and the text display area and value display area on one side of the five curve display areas; among them, the five curve display areas, from top to bottom, are: the first danger interval, the first warning interval, the normal interval, the second warning interval, and the second danger interval; the text display areas respectively display the reference names of the five curve display areas; the real-time force measurement curve is refreshed and displayed in real time in five curve display areas according to the force data fed back when the guide wire is advancing or withdrawing; the numerical display area displays real-time force data fed back when the current guide wire is being advanced or withdrawn; there are frames on the outer periphery of the five curve display areas.

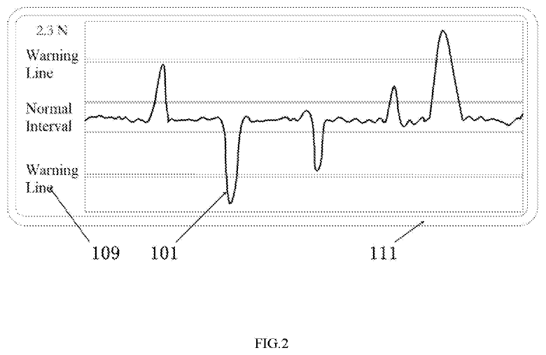

As shown in FIGS. 1-2, a real-time force measurement curve 101 is a solid line; 102 is the normal interval; there are two warning intervals on the upper and lower sides of the normal interval 102, namely the first warning interval 103 and the second warning interval 104, and the two danger intervals are the first danger interval 105 and the second danger interval 106 respectively; and two warning lines, with the first warning line 107 between the first danger interval 105 and the first warning interval 103 and the second warning line 108 between the second danger interval 106 and the second warning interval 104; the displayed value is the force data fed back when the current guide wire is being advanced or withdrawn; the explanatory text 109 can correspond to the denoting name of each display area, and is used to indicate each interval. For example, on one side of the first warning line 107 and the second warning line 108, there are corresponding warning line words to remind the doctor to pay attention to the location.

As shown in FIG. 2, the real-time force measurement curve is a curve similar to the stock trend chart. According to the needs of surgery or medical regulations, its refresh frequency can be pre-configured like 20 times per second. The ordinate of the curve is the force value, and the abscissa is the time. The curve moves 20 grids to the left every second until it reaches the leftmost side of the interval. The real-time force measurement curve is recorded and displayed with that. When the guide wire is advancing, the force measurement value is positive, the real-time force measurement curve fluctuates in the normal interval 102, the first warning interval 103, and the first danger interval 105; when the guide wire is withdrawn, the force measurement value is negative, and the real-time force measurement curve fluctuates in the normal interval 102, the second warning interval 104, and the second danger interval 106. Through the shape of the curve, the real-time force status and historical force status of the guide wire could be seen, and the doctor can grasp effective information by this.

The above-mentioned normal interval 102 represents an area of the normal fluctuation range of the force of the guide wire, that is, the safe interval. The maximum value for human safety when the guide wire is advanced and withdrawn is the upper limit and the lower limit of the safety interval 102, respectively. As long as the force measurement curve fluctuates within the normal range, the guide wire strength can be considered appropriate, which can prompt the doctor that the current guide wire is under normal force.

The warning interval is an area with two intervals, which respectively indicate the warning state when the guide wire is advanced or withdrawn. As shown in FIGS. 1-2, the first warning interval 103 is located above the normal interval 102, and the first warning interval 104 is located below the normal interval 102. When the value of the force measurement curve is larger or smaller, the curve will enter these two warning intervals. Entering this interval indicates that the force of the guide wire exceeds the normal range, but it is not yet in the danger interval. The doctor should operate with care to avoid further expansion of the force. Once the curve enters the area, the frame 111 will be displayed in the first preset mode to remind the doctor to pay attention.

Similarly, the above-mentioned dangerous interval is also an area with two intervals, which respectively represent the dangerous state when the guide wire is advanced or withdrawn. As shown in FIGS. 1-2, the first danger interval 105 is located above the first warning interval 103, and the second danger interval 106 is located below the second warning interval 104; that is, above the first warning line 107 and below the second warning line 108. When the value of the force measurement curve changes greatly, the curve will enter these two danger intervals. Entering this interval indicates that the force of the guide wire has reached the dangerous range. The doctor should combine the real-time images of the DSA to determine the state of the guide wire, adjust and reduce the force of the guide wire, and restore the curve to the normal interval. Once the curve enters the area, the frame will be displayed in the second preset mode to remind the doctor to pay attention.

In order to facilitate the doctor to quickly and accurately determine the position of the curve, the above-mentioned first preset mode and the second preset mode have different expression forms; the different expression forms may be different colors, different shapes, or different brightness.

As shown in FIGS. 1-2, the force measurement curve is placed at the top of all intervals. Below the force measurement curve, there is a normal interval 102 in the middle. Its background color is green. When the curve falls into this interval, it indicates that the force of the guide wire is normal and the operation status is safe. When the curve falls into the warning interval, it indicates that the guide wire exceeds the normal range but is not in the danger interval. The doctor should operate it carefully to avoid further expansion of the force. The frame 111 turns yellow, prompting the doctor to operate it carefully. When the curve falls into the dangerous interval, it indicates that the force of the guide wire has reached the dangerous range, and the frame 111 is displayed in red; at this time, the doctor should combine the real-time image of DSA to judge the state of the guide wire, adjust and reduce the force of the guide wire, to let the curve return to the normal range.

Taking the shape as example, when the force measurement curve is placed in the normal interval 102, the frame 111 is a thin solid line. When the curve falls into the warning interval, the frame 111 becomes a thicker dashed line; when the curve falls into the danger interval, the frame 111 becomes a thicker double dashed line to remind the doctor of the state of force at this time.

Taking brightness as example, when the force measurement curve is placed in the normal interval 102, the frame 111 is in the first brightness mode. When the curve falls into the warning interval, the frame 111 becomes the second brightness mode; when the curve falls into the dangerous interval, the frame 111 becomes the third brightness mode; the brightness values from the first brightness to the third brightness gradually increase or gradually decrease to show distinction.

As another practicable way, any two or all three of the above three manifestations can be combined to perform coordinated display, so as to remind the doctor to quickly and accurately pay attention to the stress condition and make corresponding treatment measures in time.

The force feedback display method of the interventional surgery robot provided by the embodiment of the present disclosure can intuitively present the normal, warning and dangerous states of the force of the guide wire to the doctor, which is easy to use. During the operation, by observing the change in the color, shape or brightness of the frame, an abnormality can be found the first time. In addition, the present disclosure can also be used as a modular product to be embedded in the master-end disclosure, facilitating the integration of the system.

Based on the same inventive concept, embodiments of the present disclosure also provide a force feedback display device for interventional surgery robots. Since the principle of the problem solved by the device is similar to the aforementioned force feedback display method for interventional surgery robots, the implementation of the device can refer to the aforementioned method. The implementation of the repetition will not be repeated.

The embodiment of the present disclosure provides a force feedback display device for an interventional surgery robot. The above-mentioned display method can be easily embedded in a master-end disclosure to facilitate system integration.

The display interface is very simple and easy to use. During operation, by observing the change in the color, shape or brightness of the frame, an abnormality can be found the first time.

The master-end interface of the interventional surgical robot device presents a smooth real-time force measurement curve to the doctor. By observing the high and low positions of the curve, the current force of the robot holding the guide wire could be seen, and the three states of normal, warning and dangerous states can be clearly observed. At the same time, the trend of the curve can also be used to predict the danger that may occur, so as to ensure the safe and effective operation. When the force measurement curve exceeds the normal range, the system will change the form of the frame to remind the doctor to operate carefully.

Those skilled in the art should understand that the embodiments of the present disclosure can be provided as a method, a system, or a computer program product. Therefore, the present disclosure may adopt the form of a complete hardware embodiment, a complete software embodiment, or an embodiment combining software and hardware. Moreover, the present disclosure may be in the form of a computer program product implemented on one or more computer-usable storage media (including but not limited to disk storage, optical storage, etc.) containing computer-usable program codes.

The present disclosure is described with reference to flowcharts and/or block diagrams of methods, devices (systems), and computer program products according to embodiments of the present disclosure. It should be understood that each process and/or block in the flowchart and/or block diagram, and the combination of processes and/or blocks in the flowchart and/or block diagram can be realized by computer program instructions. These computer program instructions can be provided to the processor of a general-purpose computer, a special-purpose computer, an embedded processor, or other programmable data processing equipment to generate a machine, so that the instructions executed by the processor of the computer or other programmable data processing equipment are generated It is a device that realizes the functions specified in one process or multiple processes in the flowchart and/or one block or multiple blocks in the block diagram.

These computer program instructions can also be stored in a computer-readable memory that can direct a computer or other programmable data processing equipment to work in a specific manner, so that the instructions stored in the computer-readable memory produce an article of manufacture including the instruction device. The device implements the functions specified in one process or multiple processes in the flowchart and/or one block or multiple blocks in the block diagram.

These computer program instructions can also be loaded on a computer or other programmable data processing equipment, so that a series of operation steps are executed on the computer or other programmable equipment to produce computer-implemented processing, so as to execute on the computer or other programmable equipment. Instructions provide steps for implementing functions specified in a flow or multiple flows in the flowchart and/or a block or multiple blocks in the block diagram.

Obviously, those skilled in the art can make various changes and modifications to the present disclosure without departing from the spirit and scope of the present disclosure. In this way, if these modifications and variations of the present disclosure fall within the scope of the claims of the present disclosure and their equivalent technologies, the present disclosure is also intended to include these modifications and variations.

What is claimed is:

1. A force feedback display method for an interventional surgical robot, comprising:
providing five curve display areas of real-time force measurement curve (101) in a same display screen; and
providing a text display area and a numerical display area next to the five curve display areas;
wherein the five curve display areas comprise, from top to bottom, a first danger interval, a first warning interval, a normal interval, a second warning interval and a second danger interval; the text display area correspondingly displays the names referring to the five curve display areas;
the real-time force measurement curve is refreshed and displayed in the five curve display areas in real time according to a force data fed back by a guide wire when it is advanced or withdrawn; the numerical display area displays the force data fed back by an advancing or a withdrawal of the guide wire in real time; and the outer peripheries of the five curved display regions are provided with frames.

2. The force feedback display method for an interventional surgical robot of claim 1, wherein a warning line is provided in the first danger interval and the first warning interval; and/or the warning line is provided in the second danger interval and the second warning interval.

3. The force feedback display method for an interventional surgical robot of claim 1, wherein a refresh frequency of the real-time force measurement curve displayed is pre-configured; and the ordinate of the real-time force measuring curve is the force value, and the abscissa is the time.

4. The force feedback display method for an interventional surgical robot of claim 1, wherein when the guide wire is advanced, the force measurement value displayed in the numerical display area is positive, and the real-time force measurement curve fluctuates within the normal interval, the first warning interval and the first danger interval; and
when the guide wire is withdrawn, the force measurement value displayed in the numerical display area is negative, and the real-time force measurement curve fluctuates within the normal interval, the second warning interval and the second danger interval.

5. The force feedback display method for interventional surgical robot of claim 1, wherein the normal interval represents a maximum value range of the force used to ensure the safety of human body when the guide wire is advanced or withdrawn; and the maximum value of the force used to guarantee the safety of human body when the guide wire is advanced or withdrawn is the upper and lower limits of the normal range respectively.

6. The force feedback display method for an interventional surgical robot of claim 4, wherein when the real-time force measuring curve enters the first warning interval or the second warning interval, the frame is displayed in a first preset mode; and when the real-time force measurement curve enters the first danger interval or the second danger interval, the frame is displayed in a second preset mode.

7. The force feedback display method for an interventional surgical robot of claim 6, a present form of the first preset mode is different from the second preset mode; the present form comprises different colors, shapes and/or brightness.

8. A force feedback display device for an interventional surgical robot, wherein the display is performed using the method of claim 1.
